(19) United States
(12) Patent Application Publication
(10) Pub. No.: US 2024/0293432 A1
(43) Pub. Date: Sep. 5, 2024

(54) Use of Cannabidiol to Improve Rehabilitation in Lung Cancer

(71) Applicant: UNIVERSITY OF SOUTH FLORIDA, Tampa, FL (US)

(72) Inventors: Subhra MOHAPATRA, Tampa, FL (US); Shyam MOHAPATRA, Tampa, FL (US); Ryan Green, Tampa, FL (US)

(21) Appl. No.: 18/594,989

(22) Filed: Mar. 4, 2024

Related U.S. Application Data
(60) Provisional application No. 63/488,011, filed on Mar. 2, 2023.

(51) Int. Cl. A61K 31/00; A61K 31/506; A61K 31/519; A61K 31/5377; A61P 35/00

(52) U.S. Cl. CPC A61K 31/658; A61K 31/506; A61K 31/519; A61K 31/5377; A61P 35/00

(57) ABSTRACT

A composition including a cannabinoid and an anti-cancer agent and methods of treating lung cancer using said composition.

### CROSS REFERENCE TO RELATED APPLICATIONS

This application claims the benefit of priority to U.S. Provisional Application No. 63/488,011, filed Mar. 2, 2023, which is incorporated by reference herein in its entirety.

### BACKGROUND

In 2022, an estimated 236,740 new cases of lung cancer were diagnosed in the US. There are also over 650,000 survivors of lung cancer living in the U.S. who could benefit from rehabilitation treatment. Lung cancer is the leading cause of cancer deaths. Approximately 53% of adult-onset cancer survivors report physical impairments caused by their symptoms, including fatigue, pain, and range of motion restrictions; however, only 1-2% currently receive treatment for physical impairments. Specifically, lung cancer patients are known to suffer from long-term disability. A major contributor to decreased quality-of-life in lung cancer is therapy-associated toxicity.

Cannabidiol (CBD) is a compound isolated from the Cannabis plant. CBD is generally well tolerated and sold as an over-the-counter supplement with anti-inflammatory, analgesic, and anxiolytic effects. There is a growing interest in the use of CBD to alleviate symptoms caused by cancer and/or chemotherapy including pain, sleep disruption, and anxiety. Phytochemicals, especially those that reduce inflammation and modulate oxidative stress (i.e., CBD), have been suggested as potential adjuvants to reduce cancer therapy-induced side effects. CBD has been demonstrated to reduce neuropathic pain caused by the chemotherapeutic agent paclitaxel and to protect against doxorubicin-induced cardiomyopathy in mice. CBD+tetrahydrocannabinol (THC) treatments have shown promising results in preliminary clinical trials aimed at treating cancer pain. However, overall, clinical trials of CBD to treat cancer pain have thus far yielded mixed results, and optimal treatment of individual cancer types with CBD along with optimal combinations of CBD plus specific cancer therapies and the extent to which CBD can alleviate therapy-induced toxicities are still not known. Additionally, despite the progress made, the molecular mechanisms underlying CBD's anticancer activity remain unclear.

Thus, there is a need for anticancer therapies including CBD and determinations of optimal anticancer treatments using CBD. These needs and others are at least partially satisfied by the present disclosure.

### SUMMARY

Cannabinoids, in particular CBD, can have a synergistic effect improving pain, physical function, and behavioral outcomes when combined with one or more standard-of-care cancer therapies. An analysis of transcriptomic gene expression data was used to elucidate anti-cancer mechanisms of cannabinoids (in particular, CBD) and screening of drug combinations with CBD to identify synergistic anti-cancer activity. This synergy can improve cancer rehabilitation and patient quality-of-life by reducing pain, anxiety, and tumor growth while also improving physical disabilities such as loss of mobility or stamina which can be caused both by tumors and by chemotherapy toxicity.

In an aspect, provided is a composition comprising a cannabinoid and an anti-cancer agent. In another aspect, provided is a composition comprising CBD and an EGFR inhibitor.

In another aspect, provided is a method of treating lung cancer, comprising administering a therapeutically effective amount of any of the disclosed compositions to a patient in need thereof.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 depicts pathway and function analysis of THC vs. CBD treatment induced differentially expressed genes in cancer. Differentially expressed genes in cancer cells treated with THC or CBD were analyzed for their contribution to common cellular functions in cancer using IPA (Qiagen). Effects of THC on EVSA-T breast cancer cells were compared to common effects of CBD observed in SK-N-BE (2), HepG2, MHCC97H, and primary glioma cells (NCBI GEO Database Series GSE8502, GSE151512, GSE179661, GSE57978). IPA core analysis was performed and a summary interaction network of the most significantly altered pathways is shown for CBD and THC datasets.

FIG. 2 depicts the effect of combination therapy consisting of CBD+Cisplatin or CBD+EGFR inhibitor on the viability of lung cancer cell lines H1975 and LLC as determined using CellTiter Glo viability assay (n=3). 10 μM CBD was combined with varying does of Cisplatin, Osimertinib, or Gefitinib. The IC50 of CBD alone in H1975 was 21 μM and in LLC 14.5 μM. Viability after treatment with each cancer drug was compared with and without CBD (*=p<0.05 Student's t test).

FIG. 3 depicts the effect of combination therapy including CBD+the KRAS inhibitor MRTX133. KP cells were treated with varying concentrations of CBD or MTRX133 alone or the combination of 5 μM CBD plus varying concentrations of MTRX133 for 48 hr and cell viability was measured using the CellTiterGlo assay (Promega). Representative bright field images are shown for CBD 5 μM, MTRX133 1 μM, and the combination of both drugs after 48 hr treatment. Synergy was calculated using the median effect method in CompuSyn software and the combination was found to be synergistic.

FIGS. 4A-4C depict that the behavior of mice is altered by tumor growth and by chemotherapy indicating increased pain and anxiety. C57BL/6 mice were inoculated with 5×105 LLC1 on right flank on Day 0. Cisplatin or Osimertinib were administered in doses of 10 mg/kg by intraperitoneal injection on the days indicated. DMSO+sterile saline was used as a vehicle control. Behavior was assayed using the open field test and EPM according to the timeline depicted in FIG. 4A. The open field (FIG. 4B) and EPM (FIG. 4C) tests were recorded using AnyMaze software to track animal speed, movement, and location. Significant differences between means were determined by ANOVA with Holm Sidack post hoc test p<0.05.

FIGS. 5A-5C depict the effects of CBD on behavior in lung tumor bearing mice. FIG. 5A shows IVIS (Perkin Elmer) images measuring luminescence to confirm uniform lung tumor growth on the day of group assignment. D-Luciferin (150 mg/kg) was delivered by IP injection 10 minutes prior to imaging. Vehicle or CBD (25 mg/kg) was delivered by oral gavage every alternate day and Open Field (FIG. 5B) and EPM (FIG. 5C) tests were performed. Significant differences between means were determined by ANOVA with Holm Sidack post hoc test.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “cannabis” encompasses all types of cannabis, including wild type Cannabis sativa, Cannabis chemovars, Cannabis indica, Cannabis ruderalis, and variants thereof. As used herein, the term “cannabinoid” refers to a terpene (i.e., an organic compound built from isoprene subunits) which can interact with cannabinoid receptor 1 (CB₁) or cannabinoid receptor 2 (CB₂) in a human. Cannabinoids include “phytocannabinoids,” which are produced by the cannabis plant, “endocannabinoids,” which are produced by mammals, and synthetic cannabinoids, however, the term “cannabinoid” is primarily used herein to refer to phytocannabinoids. Examples of cannabinoids include cannabinol (CBN), cannabinolic acid (CBNA), tetrahydrocannabinol (THC), tetrahydrocannabinolic acid (THCA), cannabidiol (CBD), cannabinolic acid (CBDA), tetrahydrocannabivarin (THCV), tetrahydrocannabinol acetate ester (THCOA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabidivarin (CBDV), cannabichromevarin (CBCV), cannabigerovarin (CBGV), cannabigerol monomethyl ether (CBGM), β-caryophyllene, and β-caryophyllene oxide. It is understood that reference to cannabinoids includes reference to all relevant isomers. For example, reference to CBD includes reference to all relevant isomers of CBD, including, but not limited to delta-8-CBD, delta-9-CBD, delta-10-CBD, and delta-11-CBD.

### Composition

In an aspect, provided is a composition comprising a cannabinoid and an anti-cancer agent. In some aspects, the cannabinoid can be CBN, CBNA, THC, THCA, CBD, CBDA, THCV, THCOA, CBG, CBGA, CBC, CBCA, CBL, CBLA, CBV, CBDV, CBCV, CBGV, CBGM, β-caryophyllene, β-caryophyllene oxide, or any combination thereof.

In some aspects, the cannabinoid can be non-psychoactive. In some aspects, the cannabinoid can be psychoactive. In some aspects, the cannabinoid can be CBN. In some aspects, the cannabinoid can be CBNA. In some aspects, the cannabinoid can be THC. In some aspects, the cannabinoid can be THCA. In some aspects, the cannabinoid can be CBD. In some aspects, the cannabinoid can be CBDA. In some aspects, the cannabinoid can be THCV. In some aspects, the cannabinoid can be THCOA. In some aspects, the cannabinoid can be CBG. In some aspects, the cannabinoid can be CBGA. In some aspects, the cannabinoid can be CBC. In some aspects, the cannabinoid can be CBCA. In some aspects, the cannabinoid can be CBL. In some aspects, the cannabinoid can be CBLA. In some aspects, the cannabinoid can be CBV. In some aspects, the cannabinoid can be CBDV. In some aspects, the cannabinoid can be CBCV. In some aspects, the cannabinoid can be CBGV. In some aspects, the cannabinoid can be CBGM. In some aspects, the cannabinoid can be β-caryophyllene. In some aspects, the cannabinoid can be β-caryophyllene oxide.

In some aspects, the cannabinoid can include a combination of any of the cannabinoids described above. In some aspects, the cannabinoid can include at least one non-psychoactive cannabinoid and at least one psychoactive cannabinoid. In some aspects, the cannabinoid can include CBD and THC.

In some aspects, the anti-cancer agent can be an EGFR inhibitor. In some aspects, the EGFR inhibitor can be erlotinib, osimertinib, neratinib, cetuximab, gefitinib, panitumumab, dacomitinib, lapatinib, necitumumab, mobocertinib, vandetanib, afatinib, almonertinib, brigatinib, erlotinib, icotinib, neratinib, olmutinib, pyrotinib, simotinib, or any combination thereof.

In some aspects, the EGFR inhibitor can be erlotinib. In some aspects, the EGFR inhibitor can be osimertinib. In some aspects, the EGFR inhibitor can be neratinib. In some aspects, the EGFR inhibitor can be cetuximab. In some aspects, the EGFR inhibitor can be gefitinib. In some aspects, the EGFR inhibitor can be panitumumab. In some aspects, the EGFR inhibitor can be dacomitinib. In some aspects, the EGFR inhibitor can be lapatinib. In some aspects, the EGFR inhibitor can be necitumumab. In some aspects, the EGFR inhibitor can be mobocertinib. In some aspects, the EGFR inhibitor can be vandetanib. In some aspects, the EGFR inhibitor can be afatinib. In some aspects, the EGFR inhibitor can be almonertinib. In some aspects, the EGFR inhibitor can be brigatinib. In some aspects, the EGFR inhibitor can be erlotinib. In some aspects, the EGFR inhibitor can be icotinib. In some aspects, the EGFR inhibitor can be neratinib. In some aspects, the EGFR inhibitor can be olmutinib. In some aspects, the EGFR inhibitor can be pyrotinib. In some aspects, the EGFR inhibitor can be simotinib.

In some aspects, the anti-cancer agent can be a KRAS inhibitor. In some aspects, the KRAS inhibitor can be AMG510, MRTX840, LY3499446, JAB21822, JNJ74699157, GFH925, YL15293, MRTX1133, JDQ443, or any combination thereof.

In some aspects, the KRAS inhibitor can be AMG510. In some aspects, the KRAS inhibitor can be MRTX840. In some aspects, the KRAS inhibitor can be LY3499446. In some aspects, the KRAS inhibitor can be JAB21822. In some aspects, the KRAS inhibitor can be JNJ74699157. In some aspects, the KRAS inhibitor can be GFH925. In some aspects, the KRAS inhibitor can be YL15293. In some aspects, the KRAS inhibitor can be MRTX1133. In some aspects, the KRAS inhibitor can be JDQ443.

In some aspects, the anti-cancer agent can include at least one EGFR inhibitor and at least one KRAS inhibitor. In some aspects, the anti-cancer agent can include osimertinib and MRTX1133. In some aspects, the anti-cancer agent can include gefitinib and MRTX1133.

In some aspects, the anti-cancer agent can be a chemotherapy agent. In some aspects, the at least one additional chemotherapy agent can be altretamine, bendamustine, busulfan, carboplatin, chlorambucil, cisplatin, cyclophosphamide, dacarbazine, ifosfamide, mechlorethamine, melphalan, oxaliplatin, procarbazine, temozolomide, thiotepa, trabectedin, carmustine, lomustine, streptozocin, 5-fluorouracil, 6-mercaptopurine, azacitidine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, etoposide, irinotecan, irinotecan liposomal, mitoxantrone, teniposide, topotecan, cabazitaxel, docetaxel, nab-paclitaxel, paclitaxel, vinblastine, vincristine, vincristine liposomal, vinorelbine, daunorubicin, doxorubicin, doxorubicin liposomal, epirubicin, idarubicin, mitoxantrone, valrubicin, bleomycin, dactinomycin, mitomycin-c, all-trans-retinoic acid, arsenic trioxide, asparaginase, cribulin, ixabepilone, mitotane, omacetaxine, pegaspargase, procarbazine, romidepsin, vorinostat, dexamethasone, hydrocortisone, methylprednisolone, prednisolone, prednisone, or any combination thereof.

In some aspects, the anti-cancer agent can include an EGFR inhibitor and any of the chemotherapy agents described above. In some aspects, the anti-cancer agent can include osimertinib and a chemotherapy agent. In some aspects, the anti-cancer agent can include gefitinib and a chemotherapy agent.

In some aspects, the composition can include the cannabinoid in an amount of at least about 2.5 mg/kg (e.g., at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg). In some aspects, the composition can include the cannabinoid in an amount of up to about 100 mg/kg (e.g., up to about 95 mg/kg, up to about 90 mg/kg, up to about 85 mg/kg, up to about 80 mg/kg, up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg, up to about 2.5 mg/kg).

It is considered that the composition can include the cannabinoid in an amount ranging from any of the minimum values described above to any of the maximum values described above. For example, in some aspects, the composition can include the cannabinoid in an amount of from about 2.5 mg/kg to about 100 mg/kg (e.g., from about 5 mg/kg to about 95 mg/kg, from about 10 mg/kg to about 90 mg/kg, from about 15 mg/kg to about 85 mg/kg, from about 20 mg/kg to about 80 mg/kg, from about 25 mg/kg to about 75 mg/kg, from about 30 mg/kg to about 70 mg/kg, from about 35 mg/kg to about 65 mg/kg, from about 40 mg/kg to about 60 mg/kg, from about 45 mg/kg to about 55 mg/kg, from about 2.5 mg/kg to about 50 mg/kg, from about 5 mg/kg to about 45 mg/kg, from about 10 mg/kg to about 40 mg/kg, from about 15 mg/kg to about 35 mg/kg, from about 20 mg/kg to about 30 mg/kg, from about 50 mg/kg to about 100 mg/kg, from about 55 mg/kg to about 95 mg/kg, from about 60 mg/kg to about 90 mg/kg, from about 65 mg/kg to about 85 mg/kg, from about 70 mg/kg to about 80 mg/kg).

In some aspects, the composition can include the anti-cancer agent in an amount of at least about 3 mg/kg (e.g., at least about 4 mg/kg, at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg). In some aspects, the composition can include the anti-cancer agent in an amount of up to about 80 mg/kg (e.g., up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg, up to about 4 mg/kg, up to about 3 mg/kg).

It is considered that the composition can include the anti-cancer agent in an amount ranging from any of the minimum values described above to any of the maximum values described above. For example, in some aspects, the composition can include the anti-cancer agent in an amount of from about 3 mg/kg to about 80 mg/kg (e.g., from about 4 mg/kg to about 75 mg/kg, from about 5 mg/kg to about 70 mg/kg, from about 10 mg/kg to about 65 mg/kg, from about 15 mg/kg to about 60 mg/kg, from about 20 mg/kg to about 55 mg/kg, from about 25 mg/kg to about 50 mg/kg, from about 30 mg/kg to about 45 mg/kg, from about 35 mg/kg to about 40 mg/kg, from about 3 mg/kg to about 40 mg/kg, from about 4 mg/kg to about 35 mg/kg, from about 5 mg/kg to about 30 mg/kg, from about 10 mg/kg to about 25 mg/kg, from about 15 mg/kg to about 20 mg/kg, from about 35 mg/kg to about 80 mg/kg, from about 40 mg/kg to about 75 mg/kg, from about 45 mg/kg to about 70 mg/kg, from about 50 mg/kg to about 65 mg/kg, from about 55 mg/kg to about 60 mg/kg).

In some aspects, the composition can include CBD and osimertinib. In some aspects, the composition can include CBD and gefitinib. In some aspects, the composition can include CBD and MRTX1133. In some aspects, the composition can include CBD, osimertinib, and gefitinib. In some aspects, the composition can include CBD, osimertinib, and MRTX1133. In some aspects, the composition can include CBD, gefitinib, and MRTX1133. In some aspects, the composition can include CBD, osimertinib, gefitinib, and MRTX1133.

In some aspects, the composition can include CBD in an amount of at least about 2.5 mg/kg (e.g., at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg). In some aspects, the composition can include CBD in an amount of up to about 100 mg/kg (e.g., up to about 95 mg/kg, up to about 90 mg/kg, up to about 85 mg/kg, up to about 80 mg/kg, up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg, up to about 2.5 mg/kg).

It is considered that the composition can include CBD in an amount ranging from any of the minimum values described above to any of the maximum values described above. For example, in some aspects, the composition can include CBD in an amount of from about 2.5 mg/kg to about 100 mg/kg (e.g., from about 5 mg/kg to about 95 mg/kg, from about 10 mg/kg to about 90 mg/kg, from about 15 mg/kg to about 85 mg/kg, from about 20 mg/kg to about 80 mg/kg, from about 25 mg/kg to about 75 mg/kg, from about 30 mg/kg to about 70 mg/kg, from about 35 mg/kg to about 65 mg/kg, from about 40 mg/kg to about 60 mg/kg, from about 45 mg/kg to about 55 mg/kg, from about 2.5 mg/kg to about 50 mg/kg, from about 5 mg/kg to about 45 mg/kg, from about 10 mg/kg to about 40 mg/kg, from about 15 mg/kg to about 35 mg/kg, from about 20 mg/kg to about 30 mg/kg, from about 50 mg/kg to about 100 mg/kg, from about 55 mg/kg to about 95 mg/kg, from about 60 mg/kg to about 90 mg/kg, from about 65 mg/kg to about 85 mg/kg, from about 70 mg/kg to about 80 mg/kg).

In some aspects, the composition can include osimertinib in an amount of at least about 5 mg/kg (e.g., at least about 6 mg/kg, at least about 8 mg/kg, at least about 10 mg/kg, at least about 12 mg/kg, at least about 14 mg/kg, at least about 15 mg/kg, at least about 16 mg/kg, at least about 18 mg/kg, at least about 20 mg/kg, at least about 22 mg/kg, at least about 24 mg/kg, at least about 25 mg/kg). In some aspects, the composition can include osimertinib in an amount of up to about 25 mg/kg (e.g., up to about 24 mg/kg, up to about 22 mg/kg, up to about 20 mg/kg, up to about 18 mg/kg, up to about 16 mg/kg, up to about 15 mg/kg, up to about 14 mg/kg, up to about 12 mg/kg, up to about 10 mg/kg, up to about 8 mg/kg, up to about 6 mg/kg, up to about 5 mg/kg).

It is considered that the composition can include osimertinib in an amount ranging from any of the minimum values described above to any of the maximum values described above. For example, in some aspects, the composition can include osimertinib in an amount of from about 5 mg/kg to about 25 mg/kg (e.g., from about 6 mg/kg to about 24 mg/kg, from about 8 mg/kg to about 22 mg/kg, from about 10 mg/kg to about 20 mg/kg, from about 12 mg/kg to about 18 mg/kg, from about 14 mg/kg to about 16 mg/kg, from about 5 mg/kg to about 15 mg/kg, from about 6 mg/kg to about 14 mg/kg, from about 8 mg/kg to about 12 mg/kg, from about 15 mg/kg to about 25 mg/kg, from about 16 mg/kg to about 24 mg/kg, from about 18 mg/kg to about 22 mg/kg).

In some aspects, the composition can include gefitinib in an amount of at least about 5 mg/kg (e.g., at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg). In some aspects, the composition can include gefitinib in an amount of up to about 80 mg/kg (e.g., up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg).

It is considered that the composition can include gefitinib in an amount ranging from any of the minimum values described above to any of the maximum values described above. For example, in some aspects, the composition can include gefitinib in an amount of from about 5 mg/kg to about 80 mg/kg (e.g., from about 10 mg/kg to about 75 mg/kg, from about 15 mg/kg to about 70 mg/kg, from about 20 mg/kg to about 65 mg/kg, from about 25 mg/kg to about 60 mg/kg, from about 30 mg/kg to about 55 mg/kg, from about 35 mg/kg to about 50 mg/kg, from about 40 mg/kg to about 45 mg/kg, from about 5 mg/kg to about 45 mg/kg, from about 10 mg/kg to about 40 mg/kg, from about 15 mg/kg to about 35 mg/kg, from about 20 mg/kg to about 30 mg/kg, from about 40 mg/kg to about 80 mg/kg, from about 45 mg/kg to about 75 mg/kg, from about 50 mg/kg to about 70 mg/kg, from about 55 mg/kg to about 65 mg/kg).

In some aspects, the composition can include MRTX1133 in an amount of at least about 3 mg/kg (e.g., at least about 4 mg/kg, at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg). In some aspects, the composition can include MRTX1133 in an amount of up to about 40 mg/kg (e.g., up to about 35 mg/kg, up to about 30 mg/kg, up to about 25 mg/kg, up to about 20 mg/kg, up to about 15 mg/kg, up to about 10 mg/kg, up to about 5 mg/kg, up to about 4 mg/kg, up to about 3 mg/kg).

It is considered that the composition can include MRTX1133 in an amount ranging from any of the minimum values described above to any of the maximum values described above. For example, in some aspects, the composition can include MRTX1133 in an amount of from about 3 mg/kg to about 40 mg/kg (e.g., from about 4 mg/kg to about 35 mg/kg, from about 5 mg/kg to about 30 mg/kg, from about 10 mg/kg to about 25 mg/kg, from about 15 mg/kg to about 20 mg/kg, from about 3 mg/kg to about 20 mg/kg, from about 4 mg/kg to about 15 mg/kg, from about 5 mg/kg to about 10 mg/kg, from about 15 mg/kg to about 40 mg/kg, from about 20 mg/kg to about 35 mg/kg, from about 25 mg/kg to about 30 mg/kg).

### Method

In an aspect, provided is a method of treating cancer, comprising administering a therapeutically effective amount of any of the disclosed compositions to a patient in need thereof. In some aspects, the cancer can be lung cancer.

In some aspects, the composition can be administered in an amount including the cannabinoid in an amount of at least about 2.5 mg/kg (e.g., at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg). In some aspects, the composition can be administered in an amount including the cannabinoid in an amount of up to about 100 mg/kg (e.g., up to about 95 mg/kg, up to about 90 mg/kg, up to about 85 mg/kg, up to about 80 mg/kg, up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg, up to about 2.5 mg/kg).

It is considered that the composition can be administered in an amount including the cannabinoid in an amount ranging from any of the minimum values described above to any of the maximum values described above. For example, in some aspects, the composition can be administered in an amount including the cannabinoid in an amount of from about 2.5 mg/kg to about 100 mg/kg (e.g., from about 5 mg/kg to about 95 mg/kg, from about 10 mg/kg to about 90 mg/kg, from about 15 mg/kg to about 85 mg/kg, from about 20 mg/kg to about 80 mg/kg, from about 25 mg/kg to about 75 mg/kg, from about 30 mg/kg to about 70 mg/kg, from about 35 mg/kg to about 65 mg/kg, from about 40 mg/kg to about 60 mg/kg, from about 45 mg/kg to about 55 mg/kg, from about 2.5 mg/kg to about 50 mg/kg, from about 5 mg/kg to about 45 mg/kg, from about 10 mg/kg to about 40 mg/kg, from about 15 mg/kg to about 35 mg/kg, from about 20 mg/kg to about 30 mg/kg, from about 50 mg/kg to about 100 mg/kg, from about 55 mg/kg to about 95 mg/kg, from about 60 mg/kg to about 90 mg/kg, from about 65 mg/kg to about 85 mg/kg, from about 70 mg/kg to about 80 mg/kg).

In some aspects, the composition can be administered in an amount including the anti-cancer agent in an amount of at least about 3 mg/kg (e.g., at least about 4 mg/kg, at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg). In some aspects, the composition can be administered in an amount including the anti-cancer agent in an amount of up to about 80 mg/kg (e.g., up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg, up to about 4 mg/kg, up to about 3 mg/kg).

It is considered that the composition can be administered in an amount including the anti-cancer agent in an amount ranging from any of the minimum values described above to any of the maximum values described above. For example, in some aspects, the composition can be administered in an amount including the anti-cancer agent in an amount of from about 3 mg/kg to about 80 mg/kg (e.g., from about 4 mg/kg to about 75 mg/kg, from about 5 mg/kg to about 70 mg/kg, from about 10 mg/kg to about 65 mg/kg, from about 15 mg/kg to about 60 mg/kg, from about 20 mg/kg to about 55 mg/kg, from about 25 mg/kg to about 50 mg/kg, from about 30 mg/kg to about 45 mg/kg, from about 35 mg/kg to about 40 mg/kg, from about 3 mg/kg to about 40 mg/kg, from about 4 mg/kg to about 35 mg/kg, from about 5 mg/kg to about 30 mg/kg, from about 10 mg/kg to about 25 mg/kg, from about 15 mg/kg to about 20 mg/kg, from about 35 mg/kg to about 80 mg/kg, from about 40 mg/kg to about 75 mg/kg, from about 45 mg/kg to about 70 mg/kg, from about 50 mg/kg to about 65 mg/kg, from about 55 mg/kg to about 60 mg/kg).

In some aspects, the composition can be administered in an amount including CBD in an amount of at least about 2.5 mg/kg (e.g., at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg). In some aspects, the composition can be administered in an amount including CBD in an amount of up to about 100 mg/kg (e.g., up to about 95 mg/kg, up to about 90 mg/kg, up to about 85 mg/kg, up to about 80 mg/kg, up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg, up to about 2.5 mg/kg).

It is considered that the composition can be administered in an amount including CBD in an amount ranging from any of the minimum values described above to any of the maximum values described above. For example, in some aspects, the composition can be administered in an amount including CBD in an amount of from about 2.5 mg/kg to about 100 mg/kg (e.g., from about 5 mg/kg to about 95 mg/kg, from about 10 mg/kg to about 90 mg/kg, from about 15 mg/kg to about 85 mg/kg, from about 20 mg/kg to about 80 mg/kg, from about 25 mg/kg to about 75 mg/kg, from about 30 mg/kg to about 70 mg/kg, from about 35 mg/kg to about 65 mg/kg, from about 40 mg/kg to about 60 mg/kg, from about 45 mg/kg to about 55 mg/kg, from about 2.5 mg/kg to about 50 mg/kg, from about 5 mg/kg to about 45 mg/kg, from about 10 mg/kg to about 40 mg/kg, from about 15 mg/kg to about 35 mg/kg, from about 20 mg/kg to about 30 mg/kg, from about 50 mg/kg to about 100 mg/kg, from about 55 mg/kg to about 95 mg/kg, from about 60 mg/kg to about 90 mg/kg, from about 65 mg/kg to about 85 mg/kg, from about 70 mg/kg to about 80 mg/kg).

In some aspects, the composition can be administered in an amount including the EGFR inhibitor in an amount of at least about 3 mg/kg (e.g., at least about 4 mg/kg, at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg). In some aspects, the composition can be administered in an amount including the EGFR inhibitor in an amount of up to about 80 mg/kg (e.g., up to about 75 mg/kg, up to about 70 mg/kg, up to about 65 mg/kg, up to about 60 mg/kg, up to about 55 mg/kg, up to about 50 mg/kg, up to about 45 mg/kg, up to about 40 mg/kg, up to about 35 mg/kg, up to about 30 mg/kg, up to about 25 mg/kg, up to about 20 mg/kg, up to about 15 mg/kg, up to about 10 mg/kg, up to about 5 mg/kg, up to about 4 mg/kg, up to about 3 mg/kg).

It is considered that the composition can be administered in an amount including the EGFR inhibitor in an amount ranging from any of the minimum values described above to any of the maximum values described above. For example, in some aspects, the composition can be administered in an amount including the EGFR inhibitor in an amount of from about 3 mg/kg to about 80 mg/kg (e.g., from about 4 mg/kg to about 75 mg/kg, from about 5 mg/kg to about 70 mg/kg, from about 10 mg/kg to about 65 mg/kg, from about 15 mg/kg to about 60 mg/kg, from about 20 mg/kg to about 55 mg/kg, from about 25 mg/kg to about 50 mg/kg, from about 30 mg/kg to about 45 mg/kg, from about 35 mg/kg to about 40 mg/kg, from about 3 mg/kg to about 40 mg/kg, from about 4 mg/kg to about 35 mg/kg, from about 5 mg/kg to about 30 mg/kg, from about 10 mg/kg to about 25 mg/kg, from about 15 mg/kg to about 20 mg/kg, from about 35 mg/kg to about 80 mg/kg, from about 40 mg/kg to about 75 mg/kg, from about 45 mg/kg to about 70 mg/kg, from about 50 mg/kg to about 65 mg/kg, from about 55 mg/kg to about 60 mg/kg).

In some aspects, the composition can be administered twice a day, every 1 day, every 2 days, every 3 days, every 4 days, every 5 days, every 6 days, or every 7 days.

In some aspects, the composition can include less of the anti-cancer agent than a standard dosage of the anti-cancer agent alone. In some aspects, the composition can be administered less frequently and/or for a shorter treatment duration than a standard administration of the anti-cancer agent alone. For example, in some aspects, a standard dosage of osimertinib is about 80 mg once per day, and the composition can be administered in an amount including osimertinib in an amount of from about 20 mg to about 75 mg (e.g., from about 25 mg to about 70 mg, from about 30 mg to about 65 mg, from about 35 mg to about 60 mg, from about 40 mg to about 55 mg, from about 45 mg to about 50 mg, from about 20 mg to about 50 mg, from about 25 mg to about 45 mg, from about 30 mg to about 40 mg, from about 45 mg to about 75 mg, from about 50 mg to about 70 mg, from about 55 mg to about 65 mg) twice a day, every 1 day, every 2 days, every 3 days, every 4 days, every 5 days, every 6 days, or every 7 days.

As another example, in some aspects, a standard dosage of gefitinib is about 250 mg once per day, and the composition can be administered in an amount including gefitinib in an amount of from about 50 mg to about 245 mg (e.g., from about 55 mg to about 240 mg, from about 60 mg to about 235 mg, from about 65 mg to about 230 mg, from about 70 mg to about 225 mg, from about 75 mg to about 220 mg, from about 80 mg to about 215 mg, from about 85 mg to about 210 mg, from about 90 mg to about 205 mg, from about 95 mg to about 200 mg, from about 100 mg to about 195 mg, from about 105 mg to about 190 mg, from about 110 mg to about 185 mg, from about 115 mg to about 180 mg, from about 120 mg to about 175 mg, from about 125 mg to about 170 mg, from about 130 mg to about 165 mg, from about 135 mg to about 160 mg, from about 140 mg to about 155 mg, from about 145 mg to about 150 mg, from about 50 mg to about 150 mg, from about 55 mg to about 145 mg, from about 60 mg to about 140 mg, from about 65 mg to about 135 mg, from about 70 mg to about 130 mg, from about 75 mg to about 125 mg, from about 80 mg to about 120 mg, from about 85 mg to about 115 mg, from about 90 mg to about 110 mg, from about 95 mg to about 105 mg, from about 145 mg to about 245 mg, from about 150 mg to about 240 mg, from about 155 mg to about 230 mg, from about 160 mg to about 225 mg, from about 165 mg to about 220 mg, from about 170 mg to about 215 mg, from about 175 mg to about 210 mg, from about 180 mg to about 205 mg, from about 185 mg to about 200 mg, from about 190 mg to about 195 mg) twice a day, every 1 day, every 2 days, every 3 days, every 4 days, every 5 days, every 6 days, or every 7 days.

As yet another example, in some aspects, a standard dosage of MRTX1133 is about 30 mg/kg and the composition can be administered in an amount including MRTX1133 in an amount of from about 3 mg/kg to about 40 mg/kg (e.g., from about 4 mg/kg to about 35 mg/kg, from about 5 mg/kg to about 30 mg/kg, from about 10 mg/kg to about 25 mg/kg, from about 15 mg/kg to about 20 mg/kg, from about 3 mg/kg to about 20 mg/kg, from about 4 mg/kg to about 15 mg/kg, from about 5 mg/kg to about 10 mg/kg, from about 15 mg/kg to about 40 mg/kg, from about 20 mg/kg to about 35 mg/kg, from about 25 mg/kg to about 30 mg/kg).

In some aspects, the composition can be administered via localized injection or oral administration. In some aspects, the composition is encapsulated in a nanoparticle and administered via localized injection or oral administration.

In some aspects, administration of the compound can reduce cancer cell viability. In some aspects, administration of the compound can increase the abundance of reactive oxygen species within cancer cells. In some aspects, administration of the compound can reduce nausea and anxiety in the patient. In some aspects, administration of the compound can increase mobility in the patient.

In another aspect, provided is a method of preparing an anti-cancer treatment, comprising: a) treating a cell with a cannabinoid; b) determining, via transcriptomic analysis, at least one undesirable activity caused by the cannabinoid; and c) preparing an anti-cancer treatment comprising the cannabinoid and an anti-cancer agent which has an activity that counteracts the at least one undesirable activity.

In some aspects, the at least one undesirable activity can be an upregulation or inhibition of a gene or signaling pathway. In some aspects, the at least one undesirable activity is an upregulation of EGFR signaling. In some aspects, the at least one undesirable activity is an inhibition of p53 or an upregulation of Myc oncogene.

In some aspects, the cannabinoid can be any of the cannabinoids described above or any combination of the cannabinoids described above. In some aspects, the cannabinoid can be CBD. In some aspects, the cannabinoid can be THC. In some aspects, the cannabinoid can include CBD and THC.

In some aspects, the at least one anti-cancer agent can be any of the anti-cancer agents described above. In some aspects, the at least one anti-cancer agent can be an EGFR inhibitor. In some aspects, the EGFR inhibitor can be any of the EGFR inhibitors described above. In some aspects, the EGFR inhibitor can be osimertinib. In some aspects, the EGFR inhibitor can be gefitinib. In some aspects, the EGFR inhibitor can include osimertinib and gefitinib.

### EXAMPLES

### Example 1

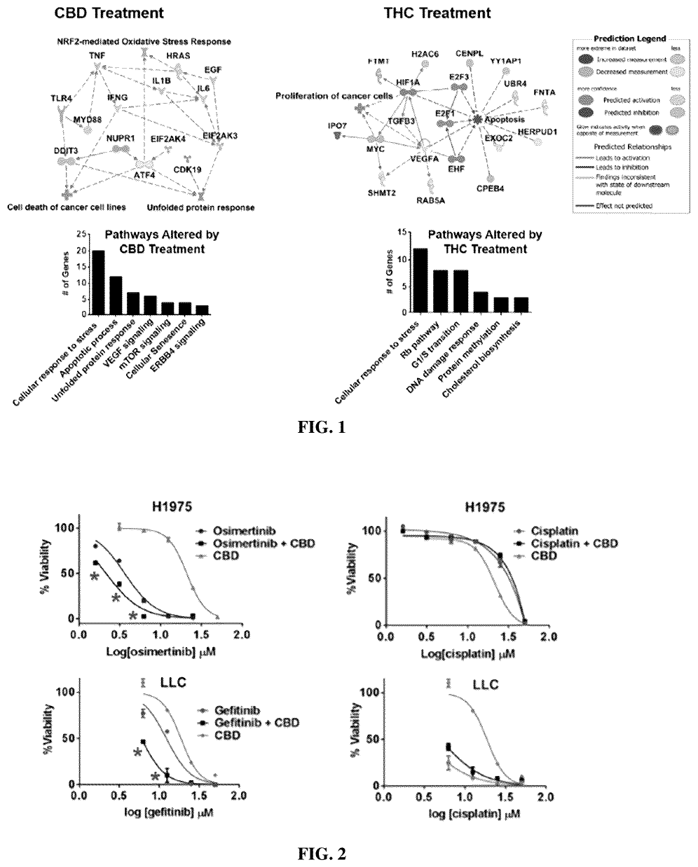

Effects of THC vs. CBD treatment on cancer cells: To begin to investigate the reason for the different effects observed between THC alone and CBD+THC in clinical trials, a study was conducted which examined transcriptomic data from CBD or THC-treated cancer cells available in the NCBI GEO database. This analysis of significantly differentially expressed genes revealed that where CBD was predicted to activate the tumor suppressor p53 and inhibit the pro-survival AKT pathway, THC was predicted to inhibit p53 and upregulate the Myc oncogene thus potentially inhibiting apoptosis. This finding is in agreement with other studies demonstrating that THC has the potential to increase the proliferation of cancer cells; however, this result is not universal as THC has also been shown to have anti-cancer effects. Thus, the effects of CBD and THC are likely somewhat dependent on the in vivo microenvironment and the specific driver mutations of the underlying tumor. Therefore, in vivo models can be used to investigate the influence of clinically relevant mutations. Furthermore, despite CBD's predicted anti-tumor effects, it was also predicted to activate epidermal growth factor receptor (EGFR) signaling which can drive cell proliferation. Therefore, it was hypothesized that CBD could have synergistic activity with EGFR inhibitors. Taken together, this analysis suggests that the combination of THC and CBD may not always be advantageous if the two compounds initiate opposing downstream signaling effects. Indeed, comparing the two data sets at the pathway level using Ingenuity Pathway Analysis revealed that CBD is predicted to activate apoptosis and inhibit proliferation in multiple cancers, whereas THC is not (FIG. 1). Therefore, more work is needed to determine the benefits of CBD treatment without THC, and thus the studies described below investigated the effects of CBD in combination with standard-of-care therapies and to optimize the rehabilitation potential of CBD in lung cancer.

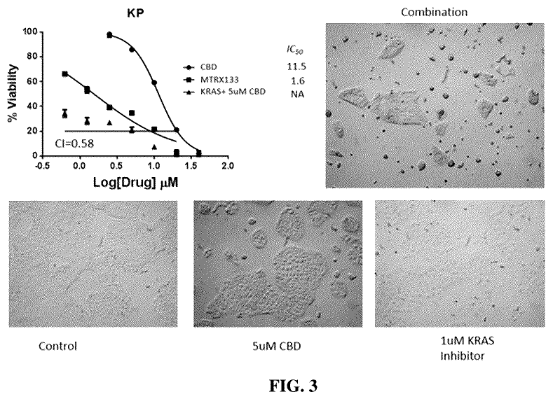

Effects of CBD combination with Standard of Care drugs in lung cancer cells: A study was conducted which screened cannabinoids, including CBD, for their ability to synergize with FDA-approved anti-cancer drugs to reduce the viability of cancer cells in vitro. Cells were treated with serial dilutions of Cisplatin, Gefitinib, or Osimertinib with or without CBD (10 μM, less than its IC₅₀), and viability was assayed after 48 hr using CellTiter Glo (Promega). IC₅₀ was calculated using GraphPad Prism (dose-response inhibition), and Combination Index (CI) was calculated using CompuSyn software. The study identified that CBD has a synergistic effect when combined with epithelial growth factor receptor (EGFR) inhibitors including Gefitinib and Osimertinib in lung cancer cell lines containing mutated or overexpressed EGFR (H1975 contains the clinically relevant T790M mutation targeted by Osimertinib) but CBD has an antagonistic or no effect on cell viability when combined with the DNA alkylating agent cisplatin (FIG. 2 and TABLE 1) To further investigate the effects of CBD treatment in lung cancer models driven by clinically relevant mutations, the study tested CBD in the KP lung cancer cell line derived from C57 BL/6 mice harboring the KRAS G12D mutation and p53 deletion. Because the major driver mutation in this cell line is known (KRAS), the study evaluated potential drug synergy between CBD and the G12D specific KRAS inhibitor MTRX1133. As shown in FIG. 3, the combination of CBD with MTRX1133 was also found to be synergistic (CI<1) in the ability to reduce cell viability following 48 hr treatment. The morphology of the cells was observed after treatment and the combination group contained many more rounded cells with an overall lower confluency.

TABLE 1
Lung cancer cell viability reduction in response to cisplatin
or EGFR inhibitors alone or in combination with CBD.

| Cell Line | Treatment | IC₅₀ (μM) | Lowest CI | Result |
|---|---|---|---|---|
| H1975 | Cisplatin | 28 | — | — |
| | CBD | 19.5 | — | — |
| | Cisplatin + CBD | 30 | 1.8 | Antagonistic |
| | Osimertinib | 3.7 | — | — |
| | Osimertinib + CBD | 2.2 | 0.47 | Synergistic |
| LLC | Cisplatin | 3.1 | — | — |
| | CBD | 19 | — | — |
| | Cisplatin + CBD | 5.0 | 2.1 | Antagonistic |
| | Gefitinib | 12.3 | — | — |
| | Gefitinib + CBD | 5.9 | 0.72 | Synergistic |

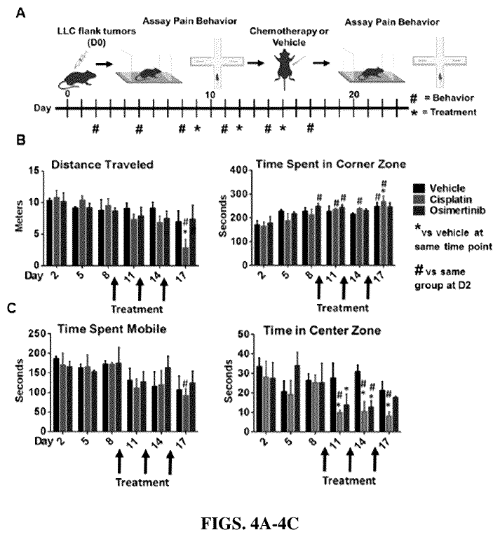

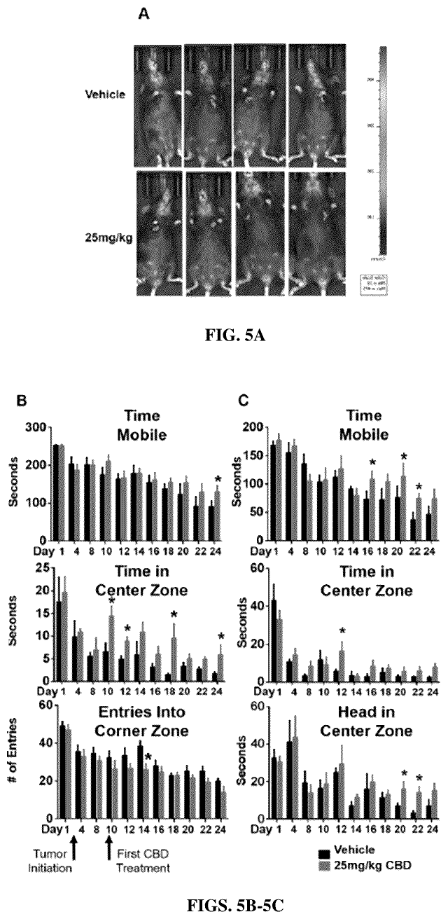

Alterations in behavior in tumor-bearing mice following chemotherapy treatment: To make a preliminary assessment of cancer and chemotherapy-derived symptoms (anxiety, reduced mobility, and pain) in mice, a study was conducted which used the syngeneic Lewis Lung Carcinoma (LLC) model of subcutaneous flank tumors. C57BL/6 mice were injected with 5×10⁵ LLC cells on the right flank. Tumor-bearing mice were treated with Cisplatin, Osimertinib, or vehicle, as outlined in FIGS. 5A-5C. The study performed the open field test (a sensory motor test used to measure general activity level) and the elevated plus maze (EPM) (test for anxiety behavior) on tumor-bearing mice throughout the period of tumor growth and treatment (FIG. 4A). In these tests, reduced physical activity was used an indicator of increased pain, and decreases in both distances traveled and time spent mobile over the tumor growth period were observed. In the open field test, greater time spent in the corner zone indicated increased anxiety. Using 60 second acclimation and 300 second test periods, an increase was observed in time spent in the corner zone from an average of 171 seconds to an average of 248 seconds between days 2 and 17 of tumor growth (FIG. 4B). In the EPM, reduced time spent in the open arms or open center zone also indicated increased anxiety. Overall, the mice spent very little time in the open arms regardless of time point or treatment group making the measure unreliable. However, mice significantly decreased their time spent in the open center zone over the course of tumor growth as well as when the vehicle group was compared to either Cisplatin or Osimertinib treatment (FIG. 4C). These measurements indicate that tumor growth was causing increased anxiety and reduced mobility in this model where pain may be a contributing factor. Also, treatment with Cisplatin appeared to yield a greater reduction in mobility compared to vehicle, and treatment with either Cisplatin or Osimertinib yielded increased anxiety compared to vehicle. One limitation of this model is the contribution of tumor location on the hind flank to decreased mobility. To overcome this limitation, future studies can use genetically engineered models of tumor growth in the lungs.

Effects of CBD on the behavior of lung tumor-bearing mice: To make a preliminary assessment of the effects of CBD on the behavior of lung tumor-bearing mice, a study was conducted which used the open field test and EPM to determine if behavioral deficiencies observed in FIGS. 4A-4C could be rescued. Lung tumors were initiated in C57BL/6 mice via intratracheal instillation of 5×105 luciferase expressing LLC cells (Perkin Elmer) and tumor growth was monitored using the Perkin Elmer In Vivo Imaging System (IVIS). Treatment groups were assigned on day 9 (FIG. 5A) to ensure each group (n=4) contained tumors of approximately the same size based on luminescence readings. CBD (25 mg/kg) or vehicle (2:1:18 parts ethanol:Tween-80:PBS) treatments by oral gavage were started on day 10 and repeated every alternate day, two hours prior to the beginning of behavioral testing. Initially, no significant change in distance travelled in the open field test was observed in the CBD group. This is consistent with previous studies of CBD treatment in naïve mice. Later as tumor growth progressed, significant increases in time mobile in both the open field and EPM were observed, which could potentially be indicative of reduced fatigue. (FIG. 5B). Furthermore, a trending decrease in number of entries into the corner zone with significant increases in time spent in the center zone was observed.

This may indicate less interest in the corner zone and, therefore, less anxiety. In the EPM (FIG. 5C), significant increases in time mobile and time the animal's head was within the open center zone were observed, indicating potential benefits to mobility and reduced anxiety. While conclusions drawn from these preliminary studies are somewhat limited due to limited statistical power and number of tests performed, and although a significant reduction in tumor growth using CBD alone was not observed at this dose, these results demonstrate that CBD has potential to lead to beneficial outcomes in an orthotopic model of lung cancer including increased mobility and decreased anxiety and that these outcomes can be modeled and assayed in mice using behavioral tests. The effects of CBD treatment in lung cancer can be investigated using additional tests to measure pain (cold plate, Von Frey, conflict avoidance) and endurance (treadmill) to discover how CBD dose and combination with cancer therapies can improve these outcomes.

Any patents, applications and publications as listed throughout this document are hereby incorporated by reference in their entirety herein.

What is claimed is:

1. A composition comprising a cannabinoid and an anti-cancer agent.

2. The composition of claim 1, wherein the cannabinoid is CBD.

3. The composition of claim 1, wherein the anti-cancer agent is an EGFR inhibitor.

4. The composition of claim 3, wherein the EGFR inhibitor is erlotinib, osimertinib, neratinib, cetuximab, gefitinib, panitumumab, dacomitinib, lapatinib, necitumumab, mobocertinib, vandetanib, afatinib, almonertinib, brigatinib, erlotinib, icotinib, neratinib, olmutinib, pyrotinib, simotinib, or any combination thereof.

5. The composition of claim 4, wherein the EGFR inhibitor is osimertinib, gefitinib, or a combination thereof.

6. The composition of claim 1, wherein the anti-cancer agent is a KRAS inhibitor.

7. The composition of claim 6, wherein the KRAS inhibitor is AMG510, MRTX840, LY3499446, JAB21822, JNJ74699157, GFH925, YL15293, MRTX1133, JDQ443, or any combination thereof.

8. The composition of claim 7, wherein the KRAS inhibitor is MRTX1133.

9. A method of treating lung cancer, comprising administering a therapeutically effective amount of the composition of claim 1 to a patient in need thereof.

10. The method of claim 9, wherein the composition is administered in an amount comprising:
the cannabinoid in an amount of from about 2.5 mg/kg to about 100 mg/kg; and
the anti-cancer agent in an amount of from about 3 mg/kg to about 80 mg/kg.

11. The method of claim 10, wherein the composition is administered twice a day, every 1 day, every 2 days, every 3 days, every 4 days, every 5 days, every 6 days, or every 7 days.

12. The method of claim 10, wherein the composition comprises less of the anti-cancer agent than a standard dosage of the anti-cancer agent.

13. The method of claim 10, wherein the composition is administered via localized injection or oral administration.

14. The method of claim 10, wherein the composition is encapsulated in a nanoparticle and administered via localized injection or oral administration.

15. A composition comprising CBD and an EGFR inhibitor.

16. The composition of claim 15, wherein the EGFR inhibitor is osimertinib, gefitinib, or a combination thereof.

17. A method of treating lung cancer, comprising administering a therapeutically effective amount of the composition of claim 15 to a patient in need thereof.

18. The method of claim 17, wherein the composition is administered in an amount comprising:
CBD in an amount of from about 5 mg/kg to about 50 mg/kg; and
the EGFR inhibitor in an amount of from about 3 mg/kg to about 80 mg/kg.

19. The method of claim 17, wherein the composition is administered via localized injection or oral administration.

20. The method of claim 17, wherein the composition is encapsulated in a nanoparticle and administered via localized injection or oral administration.
